(19) United States
(12) Patent Application Publication
(10) Pub. No.: US 2018/0231626 A1
(43) Pub. Date: Aug. 16, 2018

(54) SYSTEMS AND METHODS FOR MAGNETIC RESONANCE FINGERPRINTING FOR QUANTITATIVE BREAST IMAGING

(71) Applicant: Case Western Reserve University, Cleveland, OH (US)

(72) Inventors: Vikas Gulani, Shaker Heights, OH (US); Yong Chen, Beachwood, OH (US); Nicole Seiberlich, Shaker Heights, OH (US); Mark Griswold, Shaker Heights, OH (US)

(21) Appl. No.: 15/888,653

(22) Filed: Feb. 5, 2018

Related U.S. Application Data
(60) Provisional application No. 62/457,338, filed on Feb. 10, 2017.

(51) Int. Cl. G01R 33/48; G01R 33/50; A61B 5/055; A61B 5/00

(52) U.S. Cl. CPC G01R 33/4828; G01R 33/4824; A61B 5/4872; A61B 5/055; G01R 33/50

(57) ABSTRACT

Systems and methods for acquiring three-dimensional imaging data from a breast of a subject includes acquiring, with a nuclear magnetic resonance (NMR) system, NMR data from a volume of interest (VOI) including a breast by acquiring data in a series of variable sequence blocks. A sequence block includes one or more excitation phases, one or more readout phases, and one or more waiting phases, to cause one or more resonant species in the breast to simultaneously produce individual NMR signals. Also, at least one member of the series of variable sequence blocks differs from at least one other member of the series of variable sequence blocks in at least N sequence block parameters, N being an integer greater than one. The method also includes comparing the NMR data to a dictionary of signal evolutions from breast tissue and generating a report indicating quantitative tissue parameters over the breast.

### CROSS-REFERENCE TO RELATED APPLICATIONS

This application is based on, claims the benefit of, and incorporates herein by reference, U.S. Provisional Patent Application 62/457,338, filed Feb. 10, 2017.

### STATEMENT REGARDING FEDERALLY SPONSORED RESEARCH

This invention was made with government support under DK098503, EB011527 and HL094557 awarded by the National Institutes of Health. The government has certain rights in the invention.

### BACKGROUND

The present disclosure relates to systems and methods for magnetic resonance imaging (MRI). More particularly, the present disclosure provides systems and methods for using magnetic resonance fingerprinting (MRF) techniques to yield quantitative breast imaging information.

Conventional magnetic resonance imaging (“MRI”) pulse sequences include repetitive similar preparation phases, waiting phases, and acquisition phases that serially produce signals from which images can be made. The preparation phase determines when a signal can be acquired and determines the properties of the acquired signal. For example, a first pulse sequence may produce a T1-weighted signal at a first echo time (“TE”), while a second pulse sequence may produce a T2-weighted signal at a second TE. These conventional pulse sequences typically provide qualitative results where data are acquired with various weightings or contrasts that highlight a particular parameter (e.g., T1 relaxation, T2 relaxation).

When magnetic resonance (“MR”) images are generated, they may be viewed by a radiologist and/or surgeon who interprets the qualitative images for specific disease signatures. The radiologist may examine multiple image types (e.g., T1-weighted, T2-weighted) acquired in multiple imaging planes to make a diagnosis. The radiologist or other individual examining the qualitative images may need particular skill to be able to assess changes from session to session, from machine to machine, and from machine configuration to machine configuration.

MRI plays an important role in breast imaging for lesion detection and characterization. While MRI provides high sensitivity (approximately 90%), its specificity is relatively low (between 37% and 86%) because many conventional imaging features of benign and malignant lesions can overlap. The Breast Imaging Reporting and Data System (BIRADS) is based on morphological characteristics of the lesions and semiquantitative kinetic measurements. The qualitative nature of these factors is a limiting factor in characterization of breast tissues.

Recently, advanced functional and quantitative MR imaging techniques have emerged, such as diffusion MRI, MR spectroscopy, chemical exchange saturation transfer MRI, and sodium MR imaging. These technologies can provide biological and physiological information including cellularity, chemical composition and metabolite concentration in breast tissues. While these techniques have shown promise in preliminary clinical studies, additional quantitative imaging biomarkers are needed to further expand the quantitative space to enable definitive tissue characterization. Quantitative measurements of relaxation times are rarely performed in clinical settings because of the long scan time, especially for volumetric coverage.

T₁ and T₂ relaxation times are fundamental MRI specific properties that are determined by intrinsic tissue composition. Significantly different relaxation times have been reported in the early 1980s, for breast tumors as compared to normal tissues. Recent investigations using modern scanners also suggest that quantitative T₁ and T₂ information is beneficial for lesion detection and characterization. However, quantitative measurement of MR relaxation parameters can be technically challenging in some organs, including breast.

Recently, a new quantitative imaging framework called Magnetic resonance fingerprinting (“MRF”) was introduced, which is described, as one example, by D. Ma, et al., in “Magnetic Resonance Fingerprinting,” Nature, 2013; 495(7440):187-192. This technique allows one to characterize tissue species using nuclear magnetic resonance (“NMR”). MRF can identify different properties of a resonant species (e.g., T1 spin-lattice relaxation, T2 spin-spin relaxation, proton density) to thereby correlate this information to quantitatively assess tissue properties. Other properties like tissue types and super-position of attributes can also be identified using MRF. These properties and others may be identified simultaneously using MRF.

The development of an MRF approach for breast imaging has not been previously explored as it poses technical challenges not encountered in other applications. Most current MRF techniques generate 2D tissue property maps. For breast imaging, a MRF method with volumetric coverage is strongly preferred as breast cancers can be multicentric and multifocal. Due to the high fat content in the breasts, significant challenges from both static (B₀) and transmit (B₁) magnetic field inhomogeneities are experienced, making volumetric breast imaging technically challenging.

Thus, it would be desirable to provide systems and methods for performing quantitative analysis of breast tissue, such as to provide improved clinical tools for analyzing breast lesions that is efficient and does not require the use of ionizing radiation.

### SUMMARY OF THE INVENTION

The present invention overcomes the aforementioned drawbacks by providing systems and methods for rapid relaxometry for breast imaging using a magnetic resonance fingerprinting (MRF) technique, which allows simultaneous and volumetric quantification of tissue parameters for volumes of breast tissues.

In accordance with one aspect of the disclosure, a method is provided for acquiring three-dimensional imaging data from a breast of a subject. The method includes acquiring, with a nuclear magnetic resonance (NMR) system, NMR data from a volume of interest (VOI) including a breast by acquiring data in a series of partitions that comprise a series of variable sequence blocks. A sequence block includes one or more excitation phases, one or more readout phases, and one or more waiting phases, to cause one or more resonant species in the breast to simultaneously produce individual NMR signals. Also, at least one member of the series of variable sequence blocks differs from at least one other member of the series of variable sequence blocks in at least N sequence block parameters, N being an integer greater than one. The method also includes comparing the NMR data to a dictionary of signal evolutions from breast tissue and generating a report indicating quantitative tissue parameters over the breast.

In accordance with another aspect of the disclosure, a magnetic resonance imaging (MRI) system is provided that includes a magnet system configured to generate a polarizing magnetic field about at least a region of interest (ROI) of a subject arranged in the MRI system, a plurality of gradient coils configured to apply a gradient field to the polarizing magnetic field, and a radio frequency (RF) system configured to apply an excitation field to the subject and acquire MR image data from the ROI. The MRI system also includes a computer system programmed to control the plurality of gradient coils and the RF system to acquire imaging data from a volume of interest (VOI) including a breast by acquiring data in a series of partitions that comprise a series of variable sequence blocks. A sequence block includes one or more excitation phases, one or more readout phases, and one or more waiting phases, to cause one or more resonant species in the breast to simultaneously produce individual MRI signals. Also, at least one member of the series of variable sequence blocks differs from at least one other member of the series of variable sequence blocks in at least N sequence block parameters, N being an integer greater than one. The computer system is further programmed to compare the MRI data to a dictionary of signal evolutions from breast tissue and generate a report indicating quantitative tissue parameters over the breast.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIG. 1 is a block diagram of an example of a magnetic resonance imaging (MRI) system configured in accordance with the present disclosure.

FIG. 2 is a flow chart setting forth some examples of steps in accordance with one aspect of the present disclosure.

FIG. 3 is a schematic diagram of a pulse sequence data acquisition scheme in accordance with the present disclosure.

FIG. 4 is a non-limiting example of a flip angle pattern used in accordance with the pulse sequence of FIG. 3.

FIG. 5A is a T1 map acquired from the central partition in a phantom study performed in accordance with the present disclosure.

FIG. 5B is a T2 map acquired from the central partition in a phantom study performed in accordance with the present disclosure.

FIG. 5C is a graph showing T1 values obtained in accordance with the present disclosure compared with a standard 2D single-echo spin-echo sequence.

FIG. 5D is a graph showing T2 values obtained in accordance with the present disclosure compared with standard 2D single-echo spin-echo sequence.

FIG. 6A is a graph showing variation of phantom T1 values along the partition direction acquired in accordance with the present disclosure. Each symbol represents relaxation measurements from one vial in the phantom experiments.

FIG. 6B is a graph showing variation of phantom T2 values along the partition direction acquired in accordance with the present disclosure. Each symbol represents relaxation measurements from one vial in the phantom experiments.

FIG. 7A is a standard clinical fat-saturated image acquired from a normal subject showing substantial signal variation in the left breast (indicated by an arrow), which is likely due to B₁ field inhomogeneity.

FIG. 7B is a quantitative proton density image acquired from a normal subject performed in accordance with the present disclosure.

FIG. 7C is a quantitative T₁ image acquired from a normal subject performed in accordance with the present disclosure.

FIG. 7D is a quantitative T₂ image acquired from a normal subject performed in accordance with the present disclosure.

FIG. 8A is a set of six out of 48 total quantitative T₁ maps that were acquired from a breast of a normal subject in accordance with the present disclosure. Whole breast coverage was achieved for this subject with a total acquisition time of approximately 6 min.

FIG. 8B is a set of six out of 48 total quantitative T₂ maps that were simultaneously acquired with the T₁ maps presented in FIG. 8A.

FIG. 8C is a set of six out of 48 total quantitative M₀ maps that were simultaneously acquired with the T₁ maps presented in FIG. 8A.

FIG. 9A is a clinical post-contrast image acquired from a patient with a known IDC in the left breast.

FIG. 9B is a T2-weighted image acquired from the patient in FIG. 9A using techniques in accordance with the present disclosure.

FIG. 9C is a quantitative T₁ map acquired from the patient in FIG. 9A using techniques in accordance with the present disclosure. Longer T₁ relaxation times were observed for the tumor (T₁, 1473±103 ms) as compared to normal fibroglandular tissues in the right breast (T₁, 1198±99 ms). Prolonged T₁ relaxation times were also observed in the surrounding fibroglandular parenchyma which could be due to peritumoral tissue edema or post biopsy changes.

FIG. 9D is a quantitative T₂ map acquired from the patient in FIG. 9A using techniques in accordance with the present disclosure. Longer T₂ relaxation times were observed for the tumor (T₂, 83±5 ms) as compared to normal fibroglandular tissues in the right breast (T₂, 40±5 ms). Prolonged T₂ relaxation times were also observed in the surrounding fibroglandular parenchyma which could be due to peritumoral tissue edema or post biopsy changes.

FIG. 9E is a quantitative M₀ map acquired from the patient in FIG. 9A using techniques in accordance with the present disclosure.

### DETAILED DESCRIPTION OF THE INVENTION

Magnetic resonance fingerprinting (MRF) is a technique that facilitates mapping of tissue or other material properties based on random or pseudorandom measurements of the subject or object being imaged. In particular, MRF can be conceptualized as employing a series of varied “sequence blocks” that simultaneously produce different signal evolutions in different “resonant species” to which the RF is applied. The term “resonant species,” as used herein, refers to a material, such as water, fat, bone, muscle, soft tissue, and the like, that can be made to resonate using NMR. By way of illustration, when RF energy is applied to a volume that has both bone and muscle tissue, then both the bone and muscle tissue will produce an NMR signal. However the “bone signal” represents a first resonant species and the “muscle signal” represents a second resonant species and the two will be different. These different signals from different species can be collected simultaneously over a period of time to collect an overall “signal evolution” for the volume.

The random, pseudorandom, or otherwise varied measurements obtained in MRF techniques are achieved by varying the acquisition parameters from one repetition time (“TR”) period to the next, which creates a time series of signals with varying contrast. Examples of acquisition parameters that can be varied include flip angle (“FA”), RF pulse phase, TR, echo time (“TE’), and sampling patterns, such as by modifying one or more readout encoding gradients. The acquisition parameters are varied in a random manner, pseudorandom manner, or other manner that results in signals from different materials or tissues to be spatially incoherent, temporally incoherent, or both. In some instances, the acquisition parameters can be varied according to a non-random or a non-pseudorandom pattern that otherwise results in signals from different materials or tissues to be spatially incoherent, temporally incoherent, or both.

From these measurements, MRF processes can be designed to map a wide variety of parameters that may be mapped individually or simultaneously. Examples of such parameters include, but are not limited to, longitudinal relaxation time (T₁) transverse relaxation time (T₂), main or static magnetic field map (B₀), and proton density (PD). Unlike conventional MR systems, tissue property maps may be generated simultaneously using MRF. Thus, rather than subjecting a patient to multiple serial acquisitions that may take a half hour or more, the patient may experience a much shorter time in the bore. Similarly, rather than making a radiologist wait for multiple images that are produced serially (e.g, a first pulse sequence to generate a T₁ map, a second pulse sequence to generate a T₂ map), the radiologist may be provided with maps that are produced simultaneously from the MRF data.

Examples of such parameters include, but are not limited to, longitudinal relaxation time (T₁), transverse relaxation time (T₂), main or static magnetic field map (B₀), and proton density (PD). MRF is generally described in U.S. Pat. No. 8,723,518 and Published U.S. Patent Application No. 2015/0301141, each of which is incorporated herein by reference in its entirety.

The signal evolutions that are acquired with MRF techniques are compared with a dictionary of signal models, or templates, that have been generated for different acquisition parameters from magnetic resonance signal models, such as Bloch equation-based physics simulations. The dictionary may also comprise a series of previously acquired known evolutions. This comparison allows estimation of the physical parameters, such as those mentioned above. As an example, the comparison of the acquired signals to a dictionary are typically performed using any a matching or pattern recognition technique. The parameters for the tissue or other material in a given voxel are estimated to be the values that provide the best signal template matching. For instance, the comparison of the acquired data with the dictionary can result in the selection of a signal vector, which may constitute a weighted combination of signal vectors, from the dictionary that best corresponds to the observed signal evolution. The selected signal vector includes values for multiple different quantitative parameters, which can be extracted from the selected signal vector and used to generate the relevant quantitative parameter maps.

The stored signals and information derived from reference signal evolutions may be associated with a potentially very large data space. The data space for signal evolutions can be partially described by:

$$SE = {\sum }_{s = 1}^{{N}_{s}} {\sum }_{i = 1}^{{N}_{A}} {\sum }_{j = 1}^{{N}_{RF}} {R}_{i} ( α ) {R}_{{RF}_{ij}} ( α , φ ) R ( G ) {E}_{i} ( T 1 , T 2 , D ) {M}_{0} ; \qquad Eqn . ( 1 )$$

where SE is a signal evolution, N_{S} is a number of spins, N_{A} is a number of sequence blocks, N_{RF} is a number of RF pulses in a sequence block, α is a flip angle, Φ is a phase angle, Rᵢ(α) is a rotation due to off resonance, R_{RFij}(α, Φ) is a rotation due to RF differences, R(G) is a rotation due to a gradient, T1 is a spin-lattice relaxation, T2 is a spin-spin relaxation, D is diffusion relaxation, Eᵢ(T1, T2, D) is decay due to relaxation differences, and M₀ is the default or natural alignment to which spins align when placed in the main magnetic field.

While Eᵢ(T1, T2, D) is provided as an example, in different situations, Eᵢ(T1, T2, D) may actually be Eᵢ(T1, T2, D . . . ) or Eᵢ(T1, T2 . . . ). Also, the summation on j could be replace by a product on j.

The dictionary may store signals described by:

Sᵢ=RᵢEᵢ(Sᵢ₋₁) Eqn. (2);

where S₀ is the default or equilibrium magnetization, Sᵢ is a vector that represents the different components of magnetization Mₓ, M_{y}, M_{z} during acquisition block i, Rᵢ is a combination of rotational effects that occur during acquisition block i, and Eᵢ is a combination of effects that alter the amount of magnetization in the different states for acquisition block i. In this situation, the signal at acquisition block i is a function of the previous signal at acquisition block i−1. Additionally or alternatively, the dictionary may store signals as a function of the current relaxation and rotation effects and of previous acquisitions. Additionally or alternatively, the dictionary may store signals such that voxels have multiple resonant species or spins, and the effects may be different for every spin within a voxel. Further still, the dictionary may store signals such that voxels may have multiple resonant species or spins, and the effects may be different for spins within a voxel, and thus the signal may be a function of the effects and the previous acquisition blocks.

The present disclosure recognizes that MRF has been shown to provide rapid and simultaneous quantification of both T₁ and T₂ relaxation times. As will be described, the present disclosure provides an MRF framework for a quantitative method for 3D relaxometry in breast imaging.

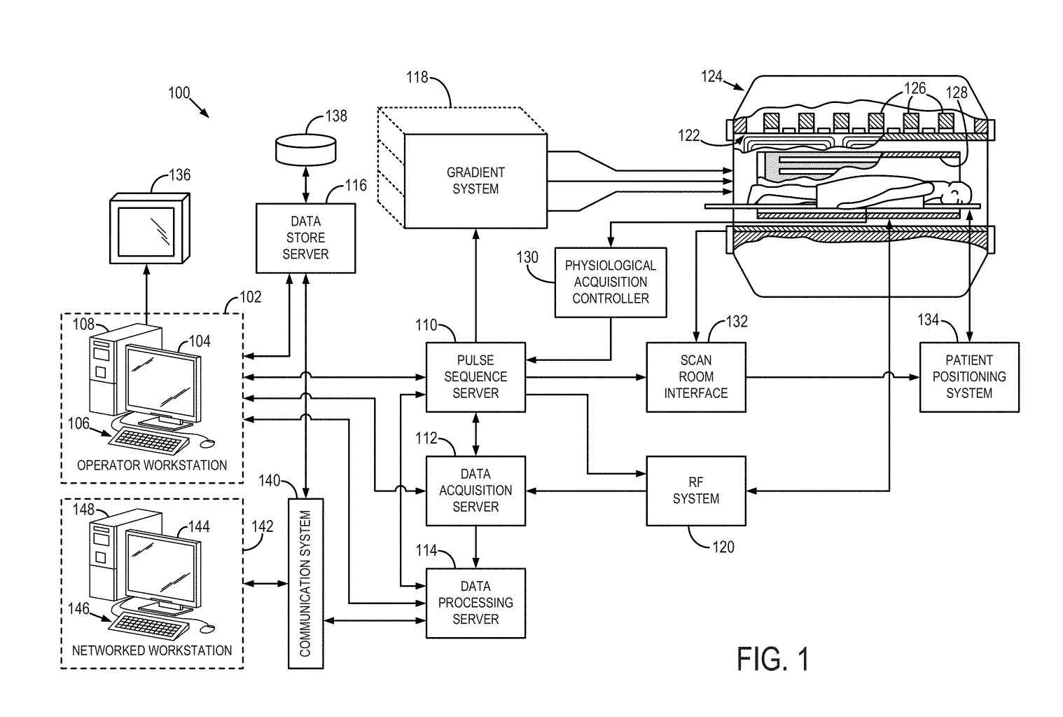

Referring particularly now to FIG. 1, an example of a system 100 that is configured to operate to acquire nuclear magnetic resonance (NMR) data. The NMR data may include magnetic resonance imaging (MRI) data, magnetic resonance spectroscopy (MRS) data, magnetic resonance fingerprinting (MRF) data, or other data, or combinations thereof. Accordingly, as used herein, the system 100 may be referred to as an NMR system, an MRI system, an MRS system, and MRF system, or the like without limitation to the type of data that the system can or does acquire or the configurations or variations on the general hardware and software of the system 100. Thus, the system 100 of FIG. 1 provides but one non-limiting example of hardware and software systems capable of performing in accordance with the present disclosure.

The system 100 includes an operator workstation 102, which typically includes a display 104; one or more input devices 106, such as a keyboard and mouse; and a processor 108. The processor 108 may include a commercially available programmable machine running a commercially available operating system. The operator workstation 102 can provide the operator interface that enables scan prescriptions to be entered into the system 100. In general, the operator workstation 102 may be coupled to four servers: a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include both proprietary or dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients G_{X}, G_{Y}, and G_{Z} used for position encoding magnetic resonance signals. The gradient coil assembly 122 forms part of a magnet assembly 124 that includes a polarizing magnet 126 and a whole-body RF coil 128 and may include a local coil.

RF waveforms are applied by the RF system 120 to the RF coil 128, or a separate local coil (not shown in FIG. 1), in order to perform the prescribed magnetic resonance pulse sequence. Responsive magnetic resonance signals detected by the RF coil 128, or a separate local coil (not shown in FIG. 1), are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. Each RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} Eqn. (3);

and the phase of the received magnetic resonance signal may also be determined according to the following relationship:

$$ϕ = {tan}^{- 1} ( \frac{Q}{I} ) . \qquad Eqn . ( 4 )$$

The pulse sequence server 110 also optionally receives patient data from a physiological acquisition controller 130. By way of example, the physiological acquisition controller 130 may receive signals from a number of different sensors connected to the patient, such as electrocardiograph (ECG) signals from electrodes, or respiratory signals from a respiratory bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (MRA) scan. By way of example, the data acquisition server 112 acquires magnetic resonance data and processes it in real-time to produce information that is used to control the scan.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 114 are conveyed back to the operator workstation 102 where they are stored. Real-time images may be output to the operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 102, or in a different facility, such as a different healthcare institution or clinic.

The networked workstation 142, whether within the same facility or in a different facility as the operator workstation 102, may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may exchange between the data processing server 114 or the data store server 116 and the networked workstations 142, such that the data or images may be remotely processed by a networked workstation 142. This data may be exchanged in any suitable format, such as in accordance with the transmission control protocol (TCP), the internet protocol (IP), or other known or suitable protocols.

The systems and methods provided herein may utilize hardware and software, such as the system 100 of FIG. 1, to perform MRF-related processes. In general, MRF techniques utilize a data acquisition scheme that causes signals from different materials or tissues to be spatially and temporally incoherent by continuously varying acquisition parameters throughout the data acquisition process. Examples of acquisition parameters that can be varied include flip angle (FA), radio frequency (RF) pulse phase, repetition time (TR), echo time (TE), sampling patterns, (such as by modifying readout encoding gradients), and the like. In typical MRF approaches, the acquisition parameters are generally varied in a random, pseudorandom manner, or otherwise varied manner.

As a result of the spatial and temporal incoherence imparted by an acquisition scheme utilizing multiple parameter values, each material or tissue is associated with a unique signal evolution or “fingerprint,” that is a function of multiple different physical parameters, including longitudinal relaxation time, T₁; transverse relaxation time, T₂; main magnetic field map, B₀; proton density, ρ, and the like.

Quantitative parameter maps are then generated from the acquired signals based on a comparison of the signals to a predefined dictionary of predicted signal evolutions. Each of these dictionaries is associated with different combinations of materials and acquisition parameters. As an example, the comparison of the acquired signals to a dictionary can be performed using any suitable matching or pattern recognition technique. This comparison results in the selection of a signal vector, which may constitute a weighted combination of signal vectors, from the dictionary that best correspond to the observed signal evolution. The selected signal vector includes values for multiple different quantitative parameters, which can be extracted from the selected signal vector and used to generate the relevant quantitative parameter maps.

As described above, the development of an MRF approach for breast imaging has not been previously explored as it poses technical challenges not encountered in other applications. In particular, breast tissues have a high fat content, which leads to significant challenges stemming from both static (B₀) and transmit (B₁) magnetic field inhomogeneities. Furthermore, current MRF techniques generate 2D tissue property maps, while volumetric coverage for breast imaging tends to be preferred as breast cancers are multicentric and multifocal. The present disclosure addresses these drawbacks by providing an MRF technique that allows for rapid relaxometry and simultaneous volumetric quantification of tissue parameters in breasts.

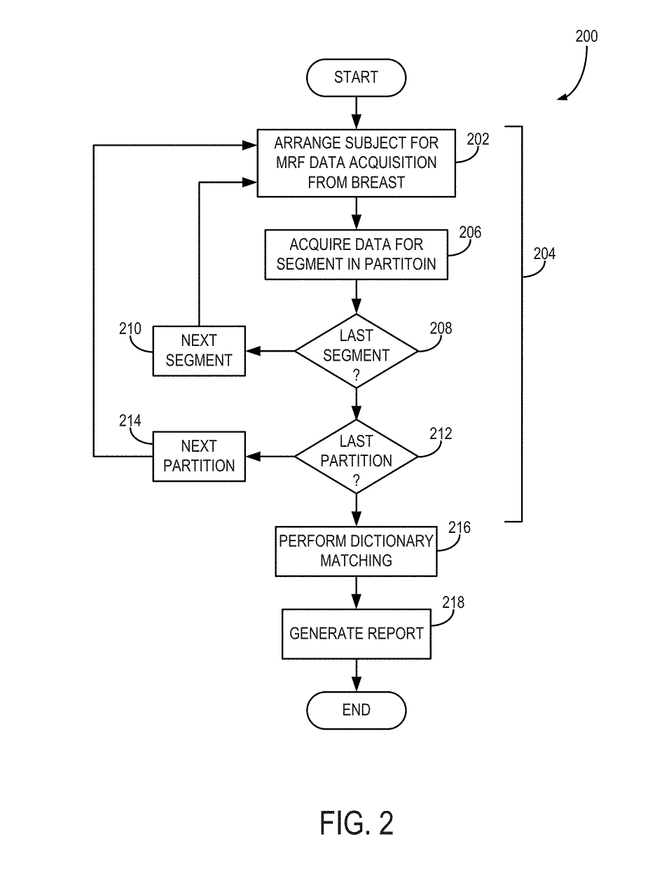

Referring to FIG. 2, a flow chart is provided that provides some, non-limiting example steps of a process 200 for performing a MRF, quantitative breast imaging process in accordance with the present disclosure, such as by using the system 100 described above with respect to FIG. 1. The process 200 begins at process block 202 by arranging the subject in a system capable of performing an MRF process to acquire MRF data from a breast of the patient, such as system 100 of FIG. 1. As indicated, generally, at 204, a pulse sequence is performed to acquire the desired MRF data such as from a patients breast. For example, the patient may be arranged in the prone position where a multi-channel breast coil may be used, such as a Siemens 3 T Verio scanner configured with eight receiver coils.

A suitable pulse sequence 204 includes a steady-state free precession (SSFP)-based pulse sequence (often referred to as a FISP-based pulse sequence by Siemens or a MPGR-based pulse sequence or even just a steady-state GRE pulse sequence by GE), such as was originally developed for 2D cardiac imaging and described by Hamilton J I, et al. Int. Soc. Magn. Reson. Med. 2015; 26, which is incorporated herein by reference in its entirety for all purposes. However, this pulse sequence may be modified for 3D breast imaging. To modify the pulse sequence for 3D breast imaging, a partition-encoding gradient is added to the 2D cardiac FISP sequence to adapt the sequence for 3D breast applications. In one aspect, the 3D breast imaging volume may be, for example, spatially encoded with phase encoding along two perpendicular spatial directions with frequency encoding along the third. The additional partition-encoding gradient allows for acquisition of three-dimensional slabs of the patient, where each slab is composed of two-dimensional partitions or slices taken along the additional partition-encoding gradient. Alternatively, in another aspect, phase encoding can be abandoned and a 3D-projection acquisition may be used, where frequency-encoding varies in three dimensions by incrementally changing the azimuthal and polar angles.

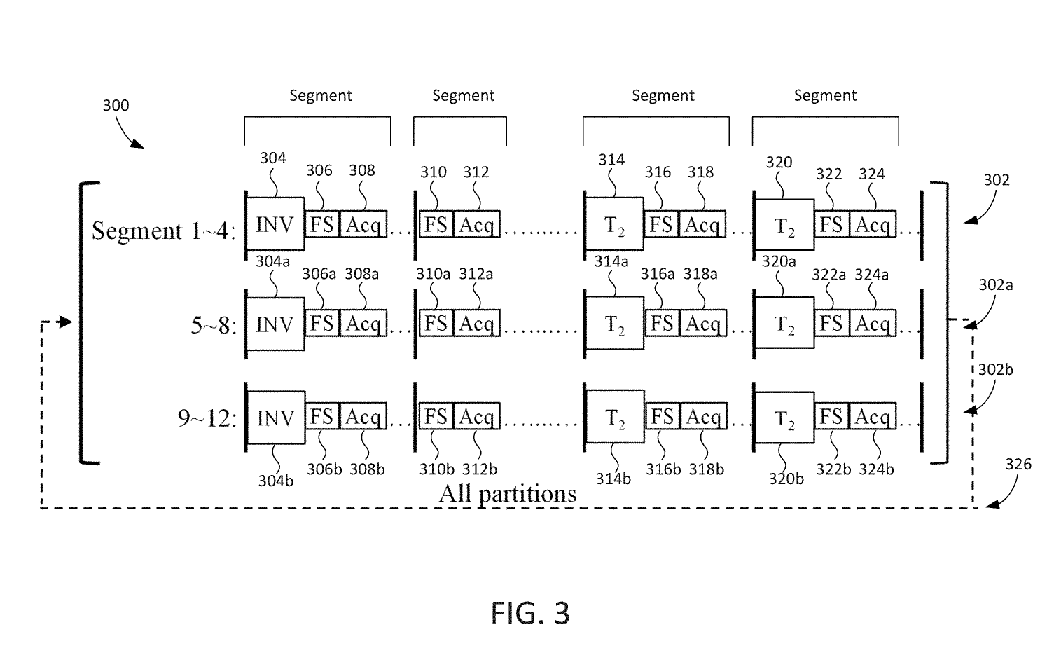

As will be further described in FIG. 3, the 3D MRF data is acquired sequentially through partitions. For each partition, the pulse sequence 204 is divided further into individual segments, where each segment includes at least one magnetization module, a data acquisition period, and a waiting period. The waiting period is applied between partitions to accommodate for longitudinal relaxation. The waiting period is further considered in the dictionary generation for accurate calculation of MRF signal evolution.

When performing the pulse sequence 204, the pulse sequence may be designed to acquire data for a given segment in a given partition at process block 206. In particular, effectuating the pulse sequence includes controlling an NMR apparatus to apply RF energy to a volume in an object being imaged. The volume may contain one or more resonant species. For example in the case of 3D breast imaging, the resonant species may include, but are not limited to, tissue, fat, water, hydrogen, and prosthetics.

The RF energy may be applied in a series of variable sequence blocks. Sequence blocks may vary in a number of parameters including, but not limited to, echo time, flip angle, phase encoding, frequency encoding, diffusion encoding, flow encoding, RF pulse amplitude, RF pulse phase, number of RF pulses, type of gradient applied between an excitation portion of a sequence block and a readout portion of a sequence block, number of gradients applied between an excitation portion of a sequence block and a readout portion of a sequence block, type of gradient applied between a readout portion of a sequence block and an excitation portion of a sequence block, number of gradients applied between a readout portion of a sequence block and an excitation portion of a sequence block, type of gradient applied during a readout portion of a sequence block, number of gradients applied during a readout portion of a sequence block, amount of RF spoiling, and amount of gradient spoiling.

Depending upon the imaging or clinical need, two, three, four, or more parameters may vary between sequence blocks. The number of parameters varied between sequence blocks may itself vary. For example, A1 (sequence block 1) may differ from A2 in five parameters, A2 may differ from A3 in seven parameters, and A3 may differ from A4 in two parameters. One skilled in the art will appreciate that there are a very-large number of series of sequence blocks that can be created by varying this large number of parameters. A series of sequence blocks can be crafted so that the series have different amounts (e.g., 1%, 2%, 5%, 10%, 50%, 99%, 100%) of unique sequence blocks as defined by their varied parameters. A series of sequence blocks may include more than ten, more than one hundred, more than one thousand, more than ten thousand, and more than one hundred thousand sequence blocks. In one example, the only difference between consecutive sequence blocks may be the number or parameters of excitation pulses.

Regardless of the particular imaging parameters that are varied or the number or type of sequence blocks, the RF energy applied at process block 202 during a sequence block is configured to cause different individual resonant species to simultaneously produce individual NMR signals or unique signal evolutions. Unlike conventional imaging techniques, in an MRF pulse sequence in accordance with the present disclosure, at least one member of the series of variable sequence blocks will differ from at least one other member of the series of variable sequence blocks in at least N sequence block parameters, N being an integer greater than one. As noted above, N may be a number greater than one. One skilled in the art will appreciate that the signal content of a signal evolution may vary directly with N. Thus, as more parameters are varied, a potentially richer signal is retrieved. Conventionally, a signal that depends on a single parameter is desired and required to facilitate imaging. Here, acquiring signals with greater information content facilitates producing more distinct and thus more matchable signal evolutions.

The pulse sequence may apply members of the series of variable sequence blocks according to a partially random or pseudo-random acquisition plan configured to under-sample the object at an under-sampling rate R. In different situations, rate R may be, for example, two, four, or greater.

Also, at process block 202, the NMR apparatus can be controlled to acquire the simultaneously produced individual NMR signals. Unlike conventional MRI imaging processes where the time during which an imaging-relevant NMR signal can be acquired is severely limited (e.g., 4-5 seconds), the NMR apparatus can be controlled to acquire NMR signal for significantly longer periods of time. For example, the NMR apparatus can be controlled to acquire signal for up to ten seconds, for up to twenty seconds, for up to one hundred seconds, or longer. NMR signals can be acquired for longer periods of time because signal information content remains viable for longer periods of time in response to the series of varied RF energy applied. In different situations, the information content in the signal evolution may remain above an information content threshold for at least five seconds, for at least ten seconds, for at least sixty seconds, or for longer. An information content threshold may describe, for example, the degree to which a subsequent signal acquisition includes information that can be retrieved and that differs from information acquired in a previous signal acquisition. For example, a signal that has no retrievable information would likely fall below an information content threshold while a signal with retrievable information that differs from information retrieved from a previous signal would likely be above the information content threshold.

After each sequence block or segment is acquired, a check is made at decision block 208 to determine if the acquired segment is the last segment. If not, at process block 210, the next segment is acquired. If so, at decision block 212, a determination of whether the current partition is the last partition is made. If not, the process moves to the next partition at process block 214, until data from the last segment of the last partition is acquired.

More particularly, MRF data may be acquired sequentially through a series of partitions 300, as shown in FIG. 3. Each partition 300 may be formed of a plurality of segments or variable sequence blocks 302-302b, such as illustrated as segments 1-4, segments 5-8, and segments 9-12. One skilled in the art would appreciate that more or less segments may be used than those illustrated in FIG. 3, for example, one could use three or more segments, four or more segments, six or more segments, eight or more segments, sixteen or more segments, twenty or more segments, or more.

For data acquisition from each segment 302-302b, a plurality of modules (e.g., 304-324, 304a-324a, 304b-324b) are performed within each partition 300. These modules generally include a magnetization preparation module (e.g., one or more excitation phases such as an inversion recovery module or T₂-preparation module), a data acquisition window, and a waiting period. In one non-limiting example, an inversion-recovery module is performed to initiate segments 1, 5, and 9. Each of the inversion-recovery modules may comprise a distinct inversion time, for example, the inversion recovery module 304 of segment 1 may have a duration of 20 ms, the inversion recovery module 304a of segment 5 may have a duration of 100 ms, and the inversion recovery module 304b of segment 9 may have a duration of 250 ms. Following each inversion recovery module 304-304b is a data acquisition module 308-308b. One challenge particular to breast imaging is the large amount of adipose tissue compared to other organs. The chemical shift between fat and water leads to image blurring when using a spiral read-out trajectory, especially when long spiral readouts are employed. To achieve improved image quality with an appropriate spatial resolution, fat suppression modules are applied in each segment to suppress the fat signal.

Segments 2, 6, and 10 may include a fat suppression module 310-310b followed by a data acquisition module 312-312b. Segments 3, 7, and 11 may include a T2 preparation module 316-316b, followed by a fat suppression module 314-314c, and a data acquisition module 318-318b. Segments 4, 8, 12, and 16 may include a T2 preparation module 316-316b, followed by a fat suppression module 314-314c, and a data acquisition module 318-318b. The T2 preparation module may use, for example, a Malcom-Levitt (MLEV) algorithm. In some forms, the fat suppression modules (306-306b, 316-316b) are configured to precede the inversion recovery modules.

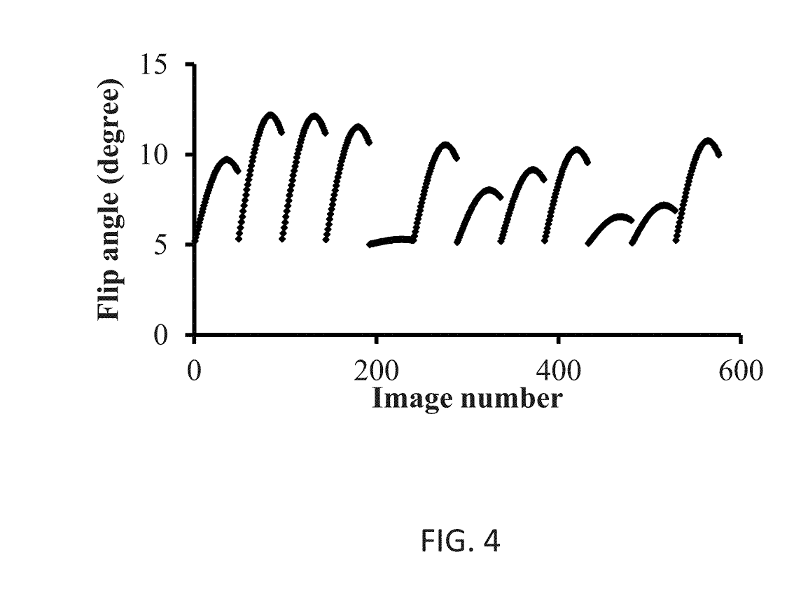

As multiple partitions 300 are acquired, the resulting data fills a 3D k-space matrix. The data acquisition modules (308-308b, 312-312b, 318-318b, 324-324b) may be configured to sample the 3D k-space matrix using a stack of projections or spirals. Following the above non-limiting example, the resulting 3D k-space data are sampled using 48 uniform-density spiral arms acquired in 48 TRs with variable flip angles ranging from 5° to 12°, as is illustrated in FIG. 4. A high in-plane reduction factor of 48 may be used, so only one spiral arm is acquired for each partition within a 3D volume. With 12 segments, a total of 576 highly undersampled volumes are acquired in one 3D MRF measurement. Golden-angle rotation may be used between spirals to further increase spatial inhomogeneity.

At the end of each segment, a variable waiting time is applied, for example, between 190 ms and 440 ms to allow longitudinal recovery for an improved SNR. In one non-limiting example, the overall duration for each segment is approximately 700 ms. Thus, as described above, for each partition 300, the data acquisition process may be divided into multiple segments (12 segments in the above-described, non-limiting example), each with a different combination of fat-saturation modules, inversion recovery modules and T2-sensitivity modules for effective T1 and T2 sensitivity.

The same or similar combination of acquisition parameters, such as the preparation modules and flip angle pattern, is repeated for each partition 300. A constant time delay 326 is implemented after each partition 300 to allow for longitudinal recovery. Suitable constant time delays 326 may be approximately 2 seconds. Other example imaging parameters used in the above non-limiting example include: a FOV=40×40 cm; a matrix size 256×256 (with an effective in-plane resolution of 1.6 mm); TR, 6.1 ms; TE, 0.9 ms; slice thickness 3 mm; number of partitions, 48; partial Fourier in the partition direction, 6/8. The overall acquisition time for 48 partitions was approximately 6 min.

The above described variable segments, or variable sequence blocks, within the partitions 300 are configured to elicit a series of spatially incoherent signal evolutions from the resonant species within the region of interest. Referring back to FIG. 2, at process block 216, the acquired spatially incoherent signal evolutions are then compared to a dictionary that includes “stored” signal evolutions therein. The “stored” or “known” signal evolutions may be generated, for example, using Bloch-like physics equations to estimate possible combinations of parameters for a T1 range, T2 range, proton density range, and the like. These three are but examples of tissue properties. Other properties or corollary properties, such as T2*, can be considered as well. In the above non-limiting example, a dictionary comprising a series of “stored” signal evolutions was calculated using Bloch equations with a wide range of T₁ and T₂ values (T₁, 100 to 3000 ms; T₂, 10 to 500 ms). In total, the dictionary contained 20,059 entries. A Singular Value Decomposition (SVD) based processing method may be implemented for efficient image reconstruction and template matching. In this instance, the SVD algorithm is applied to the MRF dictionary in the time domain to produce a low-rank approximation. In the above non-limiting example, the SVD algorithm was applied to yield 17 singular values with magnitudes larger than 0.001. Due to the linear nature of a Fourier transform, the SVD compression can be applied to the raw k-space data before gridding and taking the inverse Fourier transform.

In any case, the acquired signal in each voxel of the highly undersampled volumes (partitions) are then matched to an entry in the dictionary using pattern matching, which yields the underlying tissue parameters. Based thereon, a report is generated at process block 218. The reports may include anatomical images or maps showing the underlying tissue parameters identified from the dictionary matching at process block 216. More particularly, the report may provide quantitative tissue parameters correlated with anatomical images or maps. Alternatively, the report may simply include written text or the like that provide information on the underlying tissue, such as quantitative indications of the tissue parameters. In the instance an SVD algorithm is used to process the dictionary, the singular values, instead of the undersampled volumes, are reconstructed and matched to the compressed MRF dictionary to retrieve the underlying tissue properties. The singular values may be reconstructed using, for example, a fast non-uniform Fourier Transorm (NUFFT) toolbox.

For volumetric measurement at 3 T, significant B₁ field inhomogeneities are expected for breast imaging. To evaluate the influence of this inhomogeneity on the accuracy of T₁ and T₂ quantification using MRF, a volumetric B₁ map may be acquired in a separate scan before the MRF data is acquired. Suitable methods for generating the volumetric B₁ map include using a Bloch-Siegert method. Following acquisition, volumetric B₁ maps may be resized to match the size of the acquired MRF data. B₁ information is then incorporated into the matching algorithm to accommodate for B₁ inhomogeneity as described by Chen et al. in Radiology 2016; 279:278-286, 18, which is incorporated by reference in its entirety.

Comparing the signal evolution to one or more known, stored, simulated, and/or predicted signal evolutions can be done in a variety of fashions. For example, the “stored” or “known” signal evolutions may include previously acquired signals, simulated signals, or both. In some configurations, the stored signal evolutions may be associated with signals not acquired from the object, while in another situation the stored signal evolutions may be associated with signals acquired from the object. In configuration, the stored signals may be associated with signals acquired from the object being analyzed and signals not acquired from the object being analyzed.

The stored signals may be associated with a potentially very large data space. Thus, one skilled in the art will appreciate that the stored signal evolutions may include signals outside the set of signal evolutions characterized by:

SE=A−Be^{-t/c} Eqn. (5);

where SE is a signal evolution, A is a constant, B is a constant, t is time, and C is a single relaxation parameter.

Indeed, one skilled in the art will appreciate that the very large data space for the stored signal evolutions can be partially described by:

$$SE = {\sum }_{i = 1}^{{N}_{A}} {\sum }_{j = 1}^{{N}_{RF}} {R}_{i} ( α ) {R}_{{RF}_{ij}} ( α , ϕ ) R ( G ) {E}_{i} ( T 1 , T 2 , D ) ; \qquad Eqn . ( 6 )$$

where SE is a signal evolution, NA is a number of sequence blocks, NRF is a number of RF pulses in a sequence block, α is a flip angle, φ is a phase angle, Rᵢ(α) is a rotation due to off resonance, R_{RFij}(α, φ) is a rotation due to RF differences, R(G) is a rotation due to a gradient, T1 is spin-lattice relaxation, T2 is spin-spin relaxation, D is diffusion relaxation, and Eᵢ(T1, T2, D) is decay due to relaxation differences.

While Eᵢ(T1, T2, D) is provided as an example, one skilled in the art will appreciate that in different embodiments, Eᵢ(T1, T2, D) may actually be Eᵢ(T1, T2, D, . . . ), or Eᵢ(T1, T2, . . . ).

In one example, the summation on j could be replaced by a product on j, or the like.

In NMR, MRI, or ESR (electron spin resonance), a Bloch equation is a member of a set of macroscopic equations that are used to calculate the nuclear magnetization M=(Mx, My, Mz) as a function of time when relaxation times T1 and T2 are present. These phenomenological equations were introduced by Felix Bloch and may also be referred to as the equations of motion of nuclear magnetization. One skilled in the art will appreciate that in one embodiment Rᵢ(α), R_{RFij}(α, φ), and R(G) may be viewed as Bloch equations.

### Examples

The following examples set forth, in detail, ways in which the nuclear magnetic resonance (NMR) 100 system may be used or implemented, and will enable one of skill in the art to more readily understand the principles thereof. The following examples are presented by way of illustration and are not meant to be limiting in any way.

The present disclosure was validated using an in vivo study. The studies were approved by institutional IRB and was HIPAA compliant. Informed consent was obtained from all volunteers prior to the MRI exams. In the study, the 3D MRF method was applied to ten normal volunteers (mean age, 23.5±5.3 years; range, 18-35 years) and six patients with invasive ductal carcinoma (mean age, 52.3±10.7 years; range, 39-65 years). For each subject, a clinical fat-saturated T₂-weighted image was first acquired with a spatial resolution of 0.8×0.8×1 mm³. The 3D MRF sequence was then used in the axial plane with a spatial resolution of 1.6×1.6×3 mm³. For each patient, a clinical dynamic contrast enhanced MRI scan was also performed after the 3D MRF acquisition. The additional Bloch-Siegert B₁ measurement was performed on one asymptomatic volunteer and it was prescribed to have the same spatial coverage as the MRF scan. Region-of-interest (ROI) analysis was performed by one radiologist with 8 years of experience in breast imaging. For normal volunteers, ROIs were directly placed on the proton density maps to assess both T₁ and T₂ relaxation times for fibroglandular tissues. For patients, lesions were first identified on clinical dynamic contrast enhanced MRI exam. Based on this information, ROIs were drawn on T₂ maps corresponding to solid enhancing component of the lesion, and propagated to the T₁ maps. Note that in MRF, the T₁ maps are intrinsically registered to the T₂ maps, as the two are acquired simultaneously. T₁ and T₂ relaxation times in normal surrounding tissues were also measured from three patients, while no values were obtained from the other three patients who had almost entirely fatty breast tissue composition.

A two-tailed Student's t test was used to compare the T₁ and T₂ values obtained from ten normal subjects and six patients with IDCs. A P value of less than 0.05 was deemed significant.

The proposed method was first validated using phantoms. Agarose gel phantoms containing ten vials with different concentrations of gadolinium were used in validation. Due to the size of the vials, an MRF measurement with only 16 partitions (3 mm thickness) was performed with an in-plane resolution of 1.6 mm. The T1 and T2 relaxation times obtained with the proposed method were compared to reference T1 and T2 values acquired from the center of the vials using a 2D single-echo spin-echo sequence.

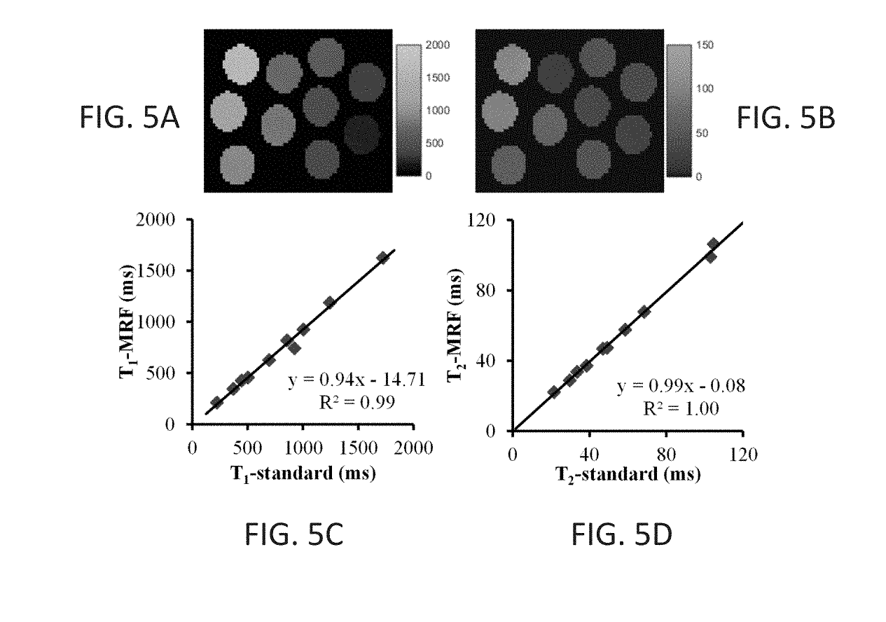

FIGS. 5(A-D) show the results of T₁ and T₂ relaxation times acquired from the central partition in a phantom experiment. A close match to the results from the reference method was observed for a large range of T₁ (200-1600 ms) and T₂ (20-105 ms) values. Compared to the reference T₁ and T₂ values acquired with the single-echo spin-echo methods, the mean percentage difference was 7.8%±4.6% and 2.6%±1.8%, for T₁ and T₂ values obtained with the 3D MRF method, respectively.

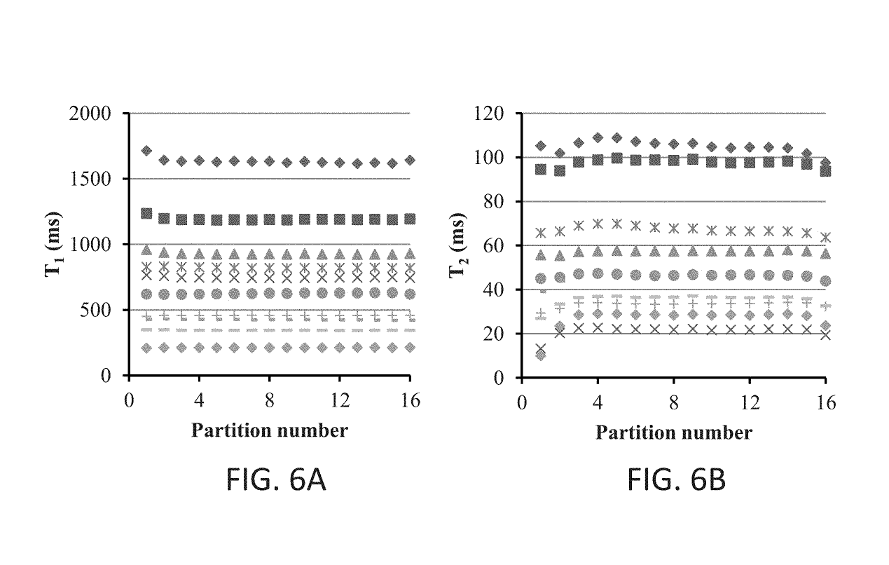

FIGS. 6(A-B) show the average T₁ and T₂ values measured from the 10 vials in all 16 partitions. Despite of a small variation in T₁ (2.6%±2.1%) and T₂ values (11.1%±14.1%) at the end partitions, a consistent measurement was observed with a T₁ variation of 0.8%±0.3% and a T₂ variation of 3.5%±2.7% across the central 14 partitions.

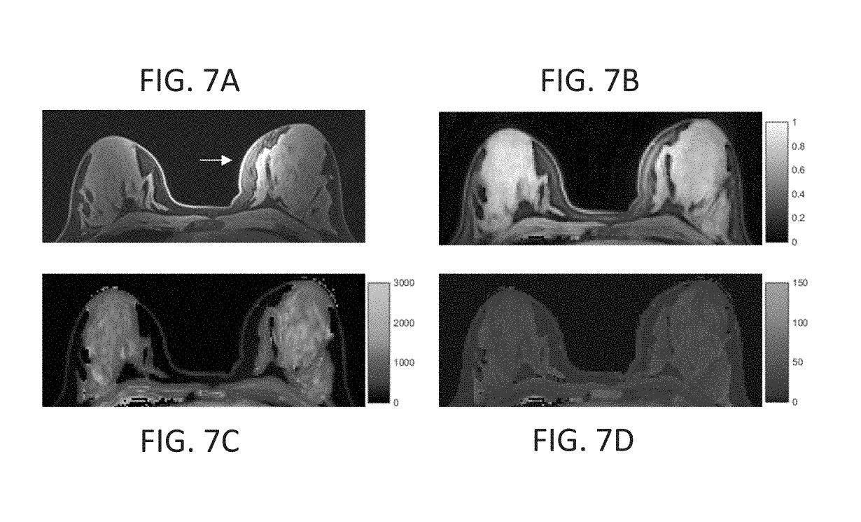

The proposed method was then validated using in vivo studies. FIGS. 7(A-D) show representative T₁ (FIG. 7C), T₂ (FIG. 7D) and proton density maps (FIG. 7B) acquired from a normal volunteer in comparison to a clinical fat-saturated image (FIG. 7A). Compared to the clinical fat-saturated image, successful fat suppression was achieved in the quantitative maps obtained with MRF. While substantial signal variation in the left breast was observed in the clinical image due to B₁ field inhomogeneity (FIG. 7A), no clear variation was noticed in the quantitative relaxation maps (FIGS. 7 B-D).

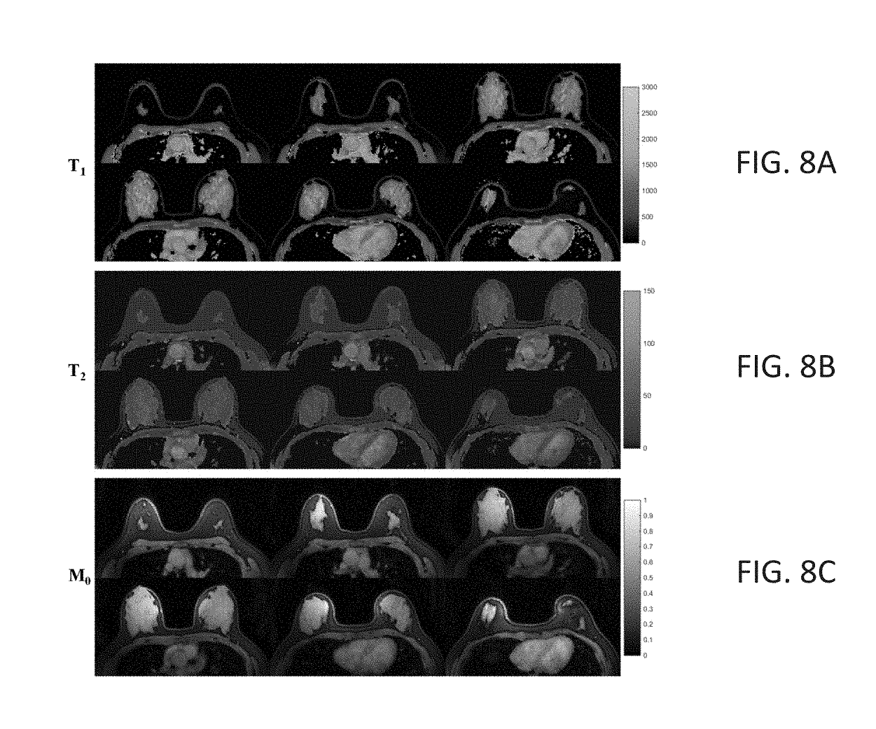

Representative 3D quantitative maps obtained from another normal volunteer are shown in FIGS. 8 (A-C). While only results from 6 partitions are depicted for ease of viewing, a total of 48 partitions were acquired in each MRF scan, which provides nearly whole breast coverage for this subject. Quantitative measurement was performed on ten normal subjects and an average T₁ of 1312±150 ms and T₂ of 48±6 ms for fibroglandular tissues were obtained, which agree well with the literature values obtained at 3 T.

The effect of B₁ field inhomogeneity on the accuracy of quantitative measurement using MRF was evaluated on a normal subject. To test the effect of B₁ field inhomogeneity, a representative 2D B₁ map from the 3D volume was acquired from the normal subject. In the 2D B₁ map approximately 20% variation in the B₁ field was observed for breast tissues in this slice, with a minimum B₁ of ˜71% encountered at the left breast and a maximum B₁ of 91% at the right breast. Quantitative maps generated without and with B₁ correction were then acquired using the proposed MRF method. With the proposed MRF method, quantitative measurement obtained without B₁ correction provides similar results as compared to those obtained in consideration of B₁ variation.

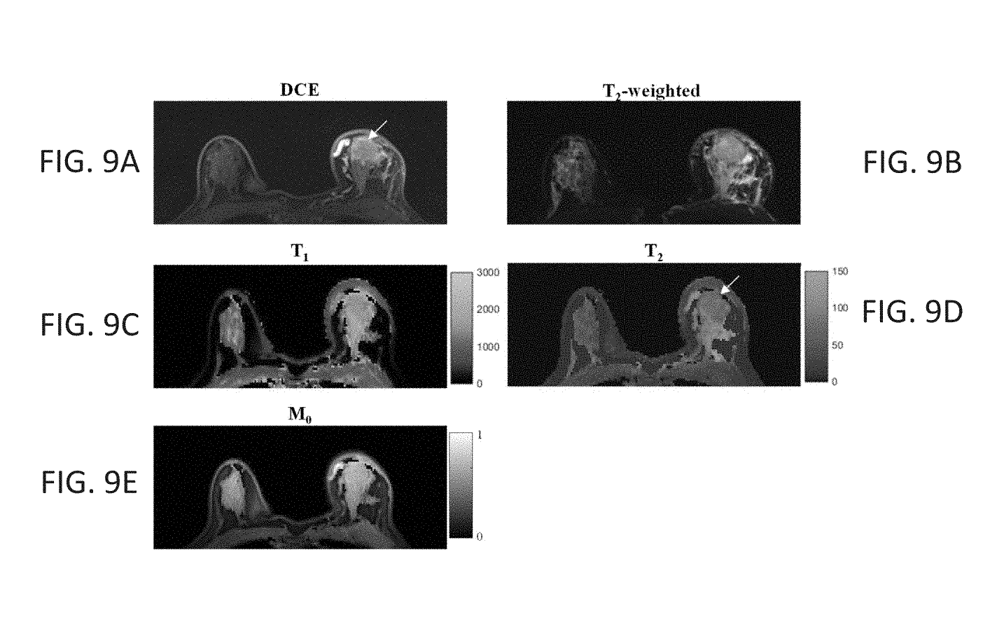

Six patients with biopsy proven invasive ductal carcinoma lesions (IDCs) were also scanned with the 3D MRF technique. FIGS. 9(A-E) show a clinical dynamic post-contrast image (FIG. 9A), and pre-contrast quantitative MRF maps such as T₂-weighted (FIG. 9B), T₁ (FIG. 9C), T₂ (FIG. 9D), and M₀ (FIG. 9E) from a patient with a tumor in the left breast. Compared to the results obtained from normal fibroglandular tissues in the right breast (T₁, 1198±99 ms; T₂, 40±5 ms), longer T₁ and T₂ relaxation times were observed for the tumor (T₁, 1473±103 ms; T₂, 83±5 ms). In addition, prolonged T₁ and T₂ relaxation times were also observed in the surrounding fibroglandular parenchyma which could be related to peritumoral tissue edema or post biopsy changes.

Dynamic post contrast images and quantitative MRF maps were also obtained using the 3D MRF technique from another patient with two IDCs in the upper outer quadrant of right breast and one benign cyst in the lower outer quadrant of left breast. Compared to the normal fibroglandular tissues in the left breast (T₁, 1184±91 ms; T₂, 42±2 ms), longer T₂ values were observed for both IDC lesions (FIG. 6a, 62±1 ms; FIG. 6b, 63±6 ms). No apparent difference was observed in T₁ values for the IDCs (FIG. 6a, 1062±123 ms; FIG. 6b, 1165±77 ms). However, much longer T₁ (1998±335 ms) and T2 (189±28 ms) relaxation times were observed for the benign cyst in the left breast.

A summary of all the T₁ and T₂ relaxation times obtained from both normal subjects and patients is presented in Table 1. The IDC values were obtained from six patients with seven total lesions. A significantly higher T₂ relaxation time was observed for IDCs, as compared to the values from either normal subjects (P<0.01) or surrounding tissues in patients (P<0.05). On the other hand, no statistical difference was noticed for T₁ relaxation time in IDCs as compared to normal breast tissues (P>0.05).

TABLE 1
Summary of T₁ and T₂ relaxation times from
both normal subjects and patients with IDC.

| | | T₁ (ms) | T₂ (ms) |
|---|---|---|---|
| Normal | | 1312 ± 150 | 48 ± 6 |
| subject | | | |
| Patient | Surrounding | 1233 ± 73 | 39 ± 4 |
| | tissue | | |
| | IDC | 1187 ± 361 | 64 ± 13 |

In this disclosure, a rapid and accurate volumetric relaxometry method was developed for breast tissue assessment using the MRF technique. The T₁ and T₂ relaxation times acquired with the proposed method are in good agreement with literature values. For example, Rakow-Penner et al. (J. Magn. Reson. Imaging 2006; 23:87-91) have reported T₁ values of 1445±93 ms and T2 values of 54±9 ms for normal fibroglandular tissue at 3 T, which match well to our findings from normal subjects. In addition, Tan et al. (Magn. Reson. Imaging 2008; 26:26-34) measured T2 relaxation times in 37 patients with IDCs using both imaging and spectroscopic methods at 1.5 T. The T2 values of 75±15 ms from imaging and 77±17 ms from spectroscopy are both slightly higher than the results of this study, but this is likely due to the fact that the measurements were performed at a lower field strength.

Transmit (B₁_{⁺}) field inhomogeneity is a well-known problem for quantitative breast imaging, especially with high-field 3 T scanners. In this disclosure, it was found that employing multiple inversion pulses with various inverison times can be used to increase the sensitivity to T₁. Moreover, using low flip angles, for example, in the range of [1° 25° ], and more particularly [5° 12° ] can help increase the sensitivity to T₁. This low flip angle pattern significantly reduces the sensitivity of the proposed technique to the B₁ field inhomogeneity. In addition, an MLEV-based T₂-preparation module was also used to minimize the adverse effects of inhomogeneous B₁ field.

One challenge particular to breast imaging is the large amount of adipose tissue compared to other organs. The chemical shift between fat and water leads to image blurring when using a spiral read-out trajectory, such as that used in MRF, especially when long spiral readouts are employed. To achieve an improved image quality with the desired spatial resolution for breast MRF, fat suppression modules may be applied to suppress fat signal. The application of fat suppression could also help improve breast cancer detection with quantitative T₂ maps such as those derived with MRF. It is well-known that fat has a longer T₂ relaxation time than that of fibroglandular tissue, and it is within the range of T₂ values for breast tumors. Removing fat information in the quantitative T2 maps could improve lesion conspicuity for better detection and characterization.

In conclusion, a rapid 3D relaxometry method was developed for breast imaging using the MRF technique. This method allows simultaneous and volumetric quantification of both T₁ and T₂ relaxation times for breast tissues.

The present invention has been described in terms of one or more preferred embodiments, and it should be appreciated that many equivalents, alternatives, variations, and modifications, aside from those expressly stated, are possible and within the scope of the invention.

1. A method for acquiring three-dimensional imaging data from a breast of a subject, the method comprising:
acquiring, with a nuclear magnetic resonance (NMR) system, NMR data from a volume of interest (VOI) including a breast by acquiring data in a series of partitions, where each partition includes a series of variable sequence blocks, where a sequence block includes one or more excitation phases, one or more readout phases, and one or more waiting phases, to cause one or more resonant species in the breast to simultaneously produce individual NMR signals and where at least one member of the series of variable sequence blocks differs from at least one other member of the series of variable sequence blocks in at least N sequence block parameters, N being an integer greater than one;
comparing the NMR data to a dictionary of signal evolutions from breast tissue; and
generating a report indicating quantitative tissue parameters over the breast.

2. The method of claim 1, wherein the one or more excitation phases in each partition includes a series of inversion recovery modules and fat suppression modules.

3. The method of claim 2, wherein the series of inversion recovery modules includes a variable inversion time.

4. The method of claim 3, wherein the variable inversion time is configured to increase between each successive inversion recovery module.

5. The method of claim 2, wherein the one or more excitation phases further comprises a series of T₂ preparation modules.

6. The method of claim 1, wherein the one or more excitation phases includes a variable flip angle.

7. The method of claim 6, wherein the variable flip angle ranges between 5° to 12°.

8. The method of claim 1, wherein the series of variable sequence blocks includes one or more variable waiting phases between sequence blocks.

9. The method of claim 8, wherein the variable waiting phases range between 190 ms and 440 ms.

10. The method of claim 1, wherein a constant delay is implemented between each partition.

11. The method of claim 1, wherein the one or more readout phases is configured to sample k-space using spiral trajectory.

12. The method of claim 5, wherein the T₂ preparation module further comprises a Malcom-Levitt algorithm.

13. The method of claim 1, further comprising diagnosing a cancerous region in the volume of interest based at least in part on the quantitative tissue parameters over the breast.

14. The method of claim 13, wherein diagnosing the cancerous region further comprises comparing T₁ and T₂ relaxation parameters over the volume of interest.

15. A magnetic resonance imaging (MRI) system comprising:
a magnet system configured to generate a polarizing magnetic field about at least a region of interest (ROI) of a subject arranged in the MRI system;
a plurality of gradient coils configured to apply a gradient field to the polarizing magnetic field;
a radio frequency (RF) system configured to apply an excitation field to the subject and acquire MR image data from the ROI;
a computer system programmed to:
control the plurality of gradient coils and the RF system to acquire imaging data from a volume of interest (VOI) including a breast by acquiring data in a series of partitions, where each partition includes a series of variable sequence blocks, where a sequence block includes one or more excitation phases, one or more readout phases, and one or more waiting phases, to cause one or more resonant species in the breast to simultaneously produce individual NMR signals and where at least one member of the series of variable sequence blocks differs from at least one other member of the series of variable sequence blocks in at least N sequence block parameters, N being an integer greater than one;
compare the MRI data to a dictionary of signal evolutions from breast tissue; and
generate a report indicating quantitative tissue parameters over the breast.

16. The MRI system of claim 15, wherein the one or more excitation phases includes a series of inversion recovery modules and fat suppression modules.

17. The MRI system of claim 15, wherein the series of inversion recovery modules includes a variable inversion time, the variable inversion time is configured to increase between each successive inversion recovery module.

18. The MRI system of claim 15, the one or more excitation phases includes a variable flip angle.

19. The MRI system of claim 15, wherein the variable flip angle ranges between 5° to 12°.

20. The MRI system of claim 15, wherein the series of variable sequence blocks includes one or more variable waiting phases between sequence blocks, where the variable waiting phases are configured to range between 190 ms and 440 ms.
